(19) United States
(12) Patent Application Publication
(10) Pub. No.: US 2010/0129856 A1
(43) Pub. Date: May 27, 2010

(54) Methods and Devices for Cell Signaling Under Pulse Stimulation

(76) Inventors: Ye Fang, Painted Post, NY (US); Vasiliy Nikolaevich Goral, Painted Post, NY (US); Joydeep Lahiri, Painted Post, NY (US); Deepti Jayaraman Mudaliar, Horseheads, NY (US); Elizabeth Tran, Painted Post, NY (US); Qi Wu, Painted Post, NY (US)

Correspondence Address: CORNING INCORPORATED, SP-TI-3-1, CORNING, NY, 14831, US

(21) Appl. No.: 12/624,062

(22) Filed: Nov. 23, 2009

Related U.S. Application Data
(60) Provisional application No. 61/117,695, filed on Nov. 25, 2008.

(51) Int. Cl. C12Q 1/02; C12M 1/34

(52) U.S. Cl. 435/29; 435/287.1

(57) ABSTRACT

The disclosure provides a device for measuring cellular responses under controlled environments. The disclosure also provides an array of devices for measuring cellular responses under controlled environments. The disclosure also provides methods to study cell signaling using the devices. By using microfluidics, the disclosure enables study of cell signaling under well-defined environment conditions. Such capability can be used for differentiating long-acting ligands from short-acting ligands, for determining the kinetics of receptor resensitization and functional recovery of receptor signaling, and for differentiating the sensitivity of a cell type to laminar flow-induced stress force. Through combination with conventional static label-free cell assays, the full spectrum of a drug compound can be studied in details, thus creating a complete representation of its pharmacology acting on living cells.

### CLAIMING BENEFIT OF PRIOR FILED U.S. APPLICATION

This application is a non-provisional application and claims the benefit of U.S. Provisional Application Ser. No. 61/117,695, filed on Nov. 25, 2008. The content of this document and the entire disclosure of other publication, patent, and patent documents mentioned herein are incorporated by reference.

### BACKGROUND

The disclosure relates generally to methods for studying cellular behavior under controlled environments, and particularly to methods using microfluidics for controlled delivery of stimulus to living cells.

The ability to examine living cells is crucial to both drug discovery and fundamental research in cell biology. Most of conventional cell-based assays involve examining of responses and behavior of living cells upon stimulation in micro-titer plates. However, cells in conventional micro-titer plates inhabit an environment that looks nothing like their natural milieu.

First, inside the human body, there are many examples of microfluidics, including little tubes and ducts and blood vessels, such as the micrometer-scale capillaries of the circulatory system and the finer, similarly sized airways in lungs. Molecules that modulate receptors and other cellular targets are commonly delivered to the targeted cells through blood streams, and/or other biofluids. The duration of stimulation with these molecules also greatly varies from cell to cell, from tissue to tissue, and from organ to organ.

Second, cell signaling is part of a complex system of communication that governs basic cellular activities and coordinates cell actions. The ability of cells to perceive and correctly respond to their microenvironment is the basis of development, tissue repair, and immunity as well as normal tissue homeostasis. Many cell signals are carried by molecules that are released by one cell and move to make contact with another cell. For example, chemical synapses, or synaptic terminals, are specialized junctions through which the cells of the nervous system signal to each other and to non-neuronal cells such as those in muscles or glands. Chemical synapses allow the neurons of the central nervous system to form interconnected neural circuits. Endocrine signals are called hormones. Hormones are produced by endocrine cells and they travel through the blood to reach all parts of the body. Specificity of signaling can be controlled if only some cells can respond to a particular hormone. Paracrine signals target only cells in the vicinity of the emitting cell. Neurotransmitters represent an example. Autocrine signals affect only cells that are of the same cell type as the emitting cell. An example for autocrine signals is found in immune cells. Juxtacrine signals are transmitted along cell membranes via protein or lipid components integral to the membrane and are capable of affecting either the emitting cell or cells immediately adjacent.

Cells receive information from their environment through a class of proteins known as receptors. While many receptors are cell surface proteins, some are found inside cells, such as estrogen receptors. Estrogen is a hydrophobic molecule that can pass through the lipid bilayer of cell surface membranes, and activates estrogen receptors. Other signaling molecules are unable to permeate the hydrophobic cell membrane due to their hydrophilic nature, so their target receptor is expressed on the membrane. When such signaling molecule activates its receptor, the signal is carried into the cell usually by means of a second messenger such as cAMP. Some signaling molecules can function as both a hormone and a neurotransmitter. For example, epinephrine and norepinephrine can function as hormones when released from the adrenal gland and are transported to the heart by way of the blood stream. Norepinephrine can also be produced by neurons to function as a neurotransmitter within the brain. Estrogen can be released by the ovary and function as a hormone or act locally via paracrine or autocrine signaling. Molecules that activate (or, in some cases, inhibit) receptors can be classified as hormones, neurotransmitters, cytokines, growth factors but all of these are called receptor ligands. The details of ligand-receptor interactions are fundamental to cell signaling.

Living cells are considered noisy or stochastic biochemical reactors. Most of the cell to cell variability is due to existence of stochastic switches or slow reaction channels involving limited numbers of reacting molecules. Stochastic switches provide inputs for amplification cascades, which translate the single molecule events into a larger population of downstream effector molecules. In eukaryotic cells, in which the number of protein or mRNA molecules is relatively large, stochastic effects primarily originate in regulation of gene activity. Another potential source of variability can be receptors activation. At low-dose stimulation, important in cell-to-cell signaling, the number of active receptors can be low enough to introduce substantial noise to downstream signaling. This high cell-to-cell variability can be one of the weapons of the immune defense. Such non-deterministic defense may be harder to overcome by relatively simple programs coded in viruses and other pathogens. Stochastic gene activation (leading to the burst of proteins) and stochastic cell activation (e.g., leading to the massive translocation) leads to “stochastic robustness” in cell regulation. If a given gene is activated, a large burst of proteins is produced, in order to assure a sufficient level of activity of these proteins. Stochastic robustness assures the minimal response to the signal. Decreasing magnitude of the signal mostly reduces the probability of response, which leads to a smaller fraction of responding cells. Stochastic robustness allows cells to respond differently to the same stimulation, but makes their individual responses better defined. Both effects could be crucial in early immune response: diversity in cell responses causes the tissue defense to be harder to overcome by relatively simple programs coded in viruses and other pathogens. The more focused single cell responses help cells to decide their individual fates such as proliferation or apoptosis.

Microfluidic devices—systems for manipulating fluids in micrometer-scale channels and wells—provide precise spatial control over reagents and samples, are capable of fast analysis times, can be automated, and can precisely manipulate picoliter volumes of material without dilution. Thus, microfluidic technology holds promise for advances in analytical biochemistry, drug discovery and development, and cellular and tissue research and engineering. For example, microfluidic systems can provide the flow velocities present in the vascular system, which is important for endothelial-cell morphology and function. However, most of microfluidic devices for cell-based studies are primarily focused on cell culturing, sorting, trapping, lysing cells and separating their contents on chips, and single cell analysis.

Thus, it would be desirable to have methods for studying the effect of molecules and compounds on living cells in a precisely controlled environment surrounding the cells.

### SUMMARY

In embodiments, there is provided a device for monitoring the response of at least one cell to pulse-stimulation, the device comprising a biosensor, the biosensor comprising a top surface, a chamber covering the biosensor, the chamber comprising an interior surface including a bottom surface, wherein the bottom surface of the chamber is, or is adjacent to, the top surface of the biosensor, at least one inlet in fluid communication with the interior surface of the chamber, the inlet comprising a valve wherein the valve is in fluid communication with at least two reservoir solutions, and at least one outlet in fluid communication with the interior surface of the chamber. The biosensor of the device may be a resonant waveguide grating biosensor, a surface plasmon resonance-based biosensor, an optical interferometer-based biosensor, or an electric biosensor. The devices may be arrayed to form a large array of devices, for example, a microtiter plate format (e.g., 4-well, 6-well, 24-well, 96-well, or 384-well microtiter plate), in which a single bottom insert containing an array of biosensors is used. In such array format, each device may have its own inlet(s) and outlet(s). Alternatively, in such array format, some or all of the device may share a common outlet interconnected each other and linked to a shared waste container, such that all expelled solutions can be drained into the shared waste container.

In embodiments there is provided a method to provide pulse stimulation to cells comprising the steps of introducing at least one cell to a chamber of a device wherein the device comprises a biosensor, the biosensor comprising a top surface, a chamber covering the biosensor, the chamber comprising an interior surface including a bottom surface, wherein the bottom surface of the chamber is or is adjacent to the top surface of the biosensor, at least one inlet in fluid communication with the interior surface of the chamber, the inlet comprising a valve wherein the valve is in fluid communication with at least two reservoir solutions, and at least one outlet in fluid communication with the interior surface of the chamber and allowing at least one cell to attach to bottom surface of the chamber. The cell is contacted with a first solution for a first amount of time, wherein the first solution flows through the chamber, contacting the cell with a second solution for a second amount of time, wherein the second solution flows through the chamber replacing the first solution and monitoring the cell response to the first and second solutions. The first solution may be a buffered assay solution with or without a compound. The second solution may contain a cell stimulatory factor, for example, an agonist for a G protein-coupled receptor, a growth factor receptor or a cell surface receptor. The cell stimulatory factor can also be any chemicals or biochemicals or biologicals or living things (e.g., virus, bacteria, or cells) that can trigger a detectable biosensor output signal in living cells.

In embodiments there is provided method for monitoring mechanical force-induced cell signaling comprising the steps of introducing at least one cell to a chamber of a device wherein the device comprises a biosensor, the biosensor comprising a top surface, a chamber covering the biosensor, the chamber comprising an interior surface including a bottom surface, wherein the bottom surface of the chamber is or is adjacent to the top surface of the biosensor, at least one inlet in fluid communication with the interior surface of the chamber, the inlet comprising a valve wherein the valve is in fluid communication with at least two reservoir solutions, and at least one outlet in fluid communication with the interior surface of the chamber, allowing at least one cell to attach to bottom surface of the chamber, contacting the cell with a first solution, wherein the first solution flows through the chamber at a stepwise increasing flow rate and monitoring the cell response to the first solution for mechanical force-induced cell signaling. The method may further comprise the steps of contacting the cell with a second solution after mechanical force-induced cell signaling is detected, wherein the second solution flows through the chamber and monitoring the cell response to the second solution. The highest flow rate of the first solution is below the threshold flow rate that causes the cell detachment. The second solution may comprise a compound, peptide, protein, hormone or other moiety.

Additional features and advantages of the disclosure will be set forth in the detailed description which follows, and in part will be readily apparent to those skilled in the art from that description or recognized by practicing the invention as described herein, including the detailed description which follows, the claims, and the appended drawings.

It is to be understood that both the foregoing general description and the following detailed description present embodiments of the invention, and are intended to provide an overview or framework for understanding the nature and character of the invention as it is claimed. The accompanying drawings are included to provide a further understanding, and are incorporated into and constitute a part of this specification. The drawings illustrate various embodiments of the invention, and together with the description serve to explain the principles and operations of the invention.

### BRIEF DESCRIPTION OF THE DRAWINGS

In embodiments:

FIG. 1A is a side view of a device for studying cellular responses;

FIG. 1B is a top view of a device for studying cellular responses;

FIG. 2A is a top view of a device for studying cellular responses;

FIG. 2B is a side view of a device for studying cellular responses;

FIG. 3 is a plot showing the characterization of a microfluidics-RWG biosensor system with regard to sensitivity under different operating regimes;

FIG. 4A is a fluorescence scan showing the cellular responses at different regions across a microfluidics-RWG biosensor system;

FIG. 4B is a fluorescence scan showing the cellular responses at different regions across a microfluidics-RWG biosensor system;

FIG. 5 is a graph showing the effect of the fluidic movement of buffer at different flow rates on the signal fluctuation of a microfluidics-RWG biosensor system having a layer of quiescent A431 cells;

FIG. 6A is a fluid dynamic simulation plot showing the flow pattern of a microfluidics-RWG biosensor system for a 30 sec. tracer injection at a flow rate of 2 microliters/min;

FIG. 6B is a fluid dynamic simulation plot showing a map of cell treatment time as a function of injection volume and flow rate;

FIG. 7A is a plot showing the influences of chemical pulse stimulation on the epinephrine-induced optical DMR response of quiescent A431 cells measured using microplate-based RWG biosensor cell assays;

FIG. 7B is a plot showing the influences of chemical pulse stimulation on the epinephrine-induced optical DMR response of quiescent A431 cells measured using microplate-based RWG biosensor cell assays;

FIG. 8A is a plot showing the DMR signals of quiescent A431 cells under pulse stimulation with 2 nM epinephrine in comparison with the epinephrine response under sustained stimulation condition of 2 nM, using the microfluidics-RWG biosensor system; and

FIG. 8B is a plot showing the DMR signals of quiescent A431 cells under pulse stimulation with 2 nM epinephrine and 100 nM salbutamol using the system;

FIG. 9A is a plot showing the DMR signal of A549 cells in response to exposure to 10 μM SFLLR-amide, followed by replacing the SFLLR-amide solution with 1×HBSS (Hank's balanced salt solution, 20 mM Hepes-KOH, pH7.1) 30 min after the SFLLR-amide stimulation, using the microfluidics-biosensor microplate system;

FIG. 9B is a plot showing the DMR signal of A549 cells in response to exposure to 5 unit/ml thrombin, followed by replacing the thrombin solution with 1×HBSS 30 min after the thrombin stimulation, using the microfluidics-biosensor microplate system;

FIG. 10A is a plot showing the DMR signal of A549 cells in response to exposure to 10 μM SFLLR-amide, followed by replacing the SFLLR-amide solution with 1×HBSS, and sequentially replacing the 1×HBSS with 10 unit/ml thrombin, using the microfluidics-biosensor microplate system; and

FIG. 10B is a plot showing the DMR signal of A549 cells in response to exposure to 5 unit/ml thrombin, followed by replacing the thrombin solution with 1×HBSS, and sequentially replacing the 1×HBSS with 10 unit/ml thrombin, using a microfluidics-biosensor microplate system;

FIG. 11 is a top view of 4×4 arrays of devices of the disclosure;

FIG. 12A is a top view of a multiwell plate system comprising a multiwell plate with a plurality of devices; and

FIG. 12B is a cut away perspective of multiwell plate system comprising a multiwell plate with a plurality of devices.

### DETAILED DESCRIPTION

Reference will now be made in detail to embodiments of the invention, and examples of which are illustrated in the accompanying drawings. Whenever possible, the same reference numerals will be used throughout the drawings to refer to the same or like parts.

The disclosure addresses the drawbacks of current cell-based assays, including label-free biosensor cellular assays, for studying cell signaling and ligand pharmacology, by way of non-limiting example. These cell-based assays are typically to use cultured cells in micro-titer plates for cell testing. The stimulation of cells in these assays is generally global stimulation—receptors or cellular targets in the cells are un-selectively modulated by the molecules introduced. Also, the stimulation mostly takes place under equilibrium conditions. Furthermore, the stimulation takes place a specific period of times; depending on the cellular events downstream the target, the assay time could be seconds (e.g., Ca²⁺ mobilization), minutes (e.g., changes in cyclic AMP (cAMP) level, or kinase phosphorylation states), or hours (e.g., gene expression). The compound is generally introduced once without removal, thus creating a sustained period of stimulation.

The disclosure discloses a device for measuring cellular responses under controlled environments. The disclosure also provides an array of such devices. The disclosure also provides methods to study cell signaling using the devices of the present system. By using microfluidics or fluidics, the disclosure enables study of cell signaling under well-defined environment conditions. Such capability can be used for differentiating long-acting ligands from short-acting ligands, for determining the kinetics of receptor resensitization and functional recovery of receptor signaling, for differentiating functional selectivity of agonists, and for differentiating the sensitivity of a cell type to laminar flow-induced stress force. Through combination with conventional static label-free cell assays, the full spectrum of a drug compound can be studied in details, thus creating a complete representation of its pharmacology acting on living cells.

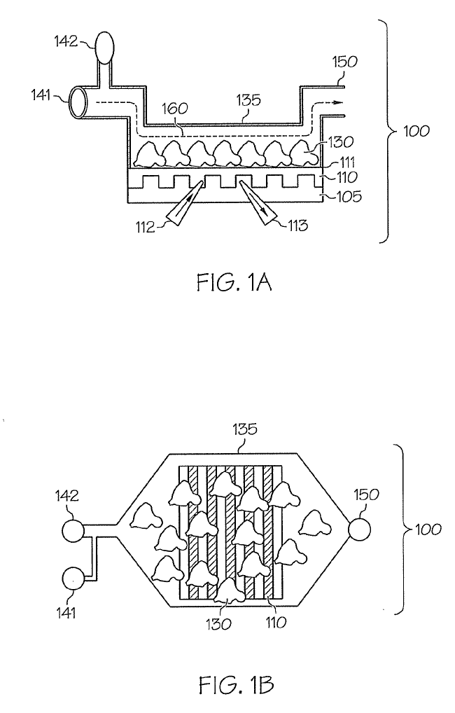

In embodiments, there is provided a device 100 comprising a biosensor, the biosensor comprising a top surface and a chamber 135 covering the biosensor (FIGS. 1a and 1b). The chamber 135 may comprise an interior surface wherein the interior surface comprises a bottom surface. The bottom surface 111 may be adjacent to or is the top surface of the biosensor. The biosensor, for example, may be a resonant waveguide grating biosensor, a surface plasmon resonance-based biosensor, an optical interferometer-based biosensor, or an electric biosensor. The chamber 135 may comprise at least two inlets 141 and 142, respectively, in fluid communication with the interior surface of the chamber and where each is independently in fluid communication with reservoir solution. The chamber 135 further comprises at least one outlet 150. When a resonant waveguide grating biosensor is used as illustrated in FIGS. 1a and 1b, the biosensor may comprise, for example, a substrate made of glass or plastic 105 within which a grating structure may be embedded, a waveguide 110 thin film, a light source 112 and a means to detect and process the resulting refracted light 113. For optical based biosensors, only the mass redistribution within the detection zone and the bottom portion of cells 130 is directly measured. Cells 130 may be directly cultured onto the bottom surface of the chamber 135, or brought to contact with the bottom surface of the chamber 135. Micropumps may be used to generate fluidic flow 160 within the chamber of the device 100.

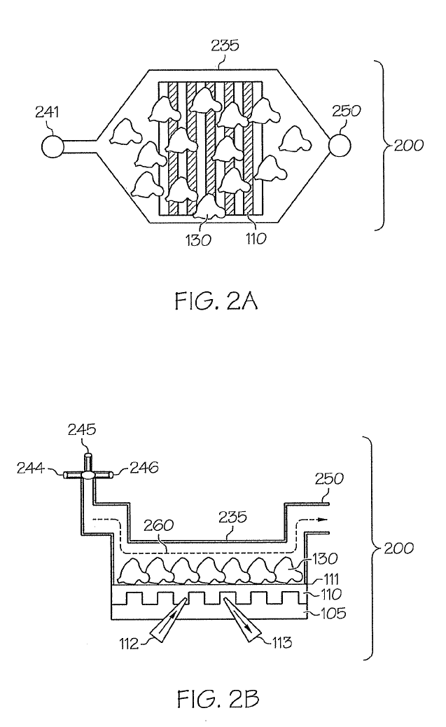

In embodiments, there is provided a device 200 comprising a biosensor, the biosensor comprising a top surface, and a chamber 235 covering the biosensor, as illustrated in FIGS. 2a and 2b. The chamber 235 may comprise an interior surface, wherein the interior surface comprises a bottom surface. The bottom surface 111 of the chamber 235 may be adjacent to or is the top surface of the biosensor. The chamber 235 may comprise at least one inlet 241 in fluid communication with the interior surface of the chamber and outlet 250 in fluid communication with the interior surface of the chamber. The inlet 241 may further comprise a valve 242 that may be connected with or attached to or built within the inlet 241. The valve 242 may be in fluid communication with at least one reservoir solution through at least one tube or channel. By way of non-limiting example, FIG. 2a shows valve 242 connected to three tubes or channels 244, 245 and 246. It will be appreciated that any number of tubes may be attached to valve 242 depending on the desired use for the device 200. Valve 242 may be connected between the inlet 241 and at least one tube connecting with a solution reservoir (i.e. 244, 245 and 246). Micropumps may be used to generate through the valve/inlet a fluidic flow 260 within the channel of the device 200.

The terms microfluidics and fluidics refer to the dimension of the biosensor system where laminar flow takes place. It should be noted that although the term fluidics or microfluidics may be used herein, it is meant to refer to both. The microfluidics generally deals with the behavior, precise control and manipulation of fluids that are geometrically constrained to a small, typically sub-millimeter scale. In embodiments of the disclosure, the chambers may be about 50 micrometers to several millimeters in height. The disclosure provides methods for examination of living cells under microfluidics or fluidics. Since the living cells are typically large (about several microns to tens of microns in diameter), the chambers of the devices 100 or 200 may be large in both width and height and may vary based on the biosensor utilized, the amount of sample required and other considerations such as, but not limited to, optimal cell culture conditions for the cell type examined. In an exemplary embodiment, the width of the chamber may be from about 100 microns to about 10 millimeters, or from about 500 microns to about 5 millimeters. Alternately, the height may be from about 1 millimeter to about 5 millimeters or from about 2 to about 10 millimeters. In an alternate exemplary embodiment, the height may be from about 50 microns to 5 millimeters or from about 200 microns to about 2 millimeters. Alternatively, the width may be from about 100 microns to about 500 microns or from about 200 microns to about 1 millimeter. With the above ranges for the geometries of the chambers, the Reynolds number, which characterizes the presence of turbulent flow, is extremely low and therefore the flow may remain laminar. Thus, two fluids joining together will not mix readily via turbulence, so diffusion alone must cause the two fluids to mingle. It should be noted that the laminar flow generated using typical microfluidics is helpful, but not required for the disclosure. In fact in some embodiments, it may be helpful to eliminate laminar flow through proper control of the height of chamber and flow of solution. The desired dimensions of the chambers may be determined by the skilled artisan, based on the desired use of the device, without undue experimentation. Therefore, the above ranges are only given as a guide and are in no way meant to be limited.

In embodiments, the devices 100 and/or 200 may comprise a plurality of chambers 135 and/or 235, where devices 100 and/or 200 each independently comprise at least two inlets and an outlet or alternatively, at least two devices 100 and/or 200 share an inlet and/or outlet. The system may also be arranged such that valves 142 and/or 242 may be used to control flow between two or more chambers. The microfluidics-biosensor array system may have a format compatible to standard Society for Biomolecular Screening (SBS) microliter plates (e.g., 4-well, 6-well, 24-well, 96-well, or 384-well microtiter plate), in which a single bottom insert (i.e., the bottom portion of the microtiter plate without the wells) containing an array of biosensors is used.

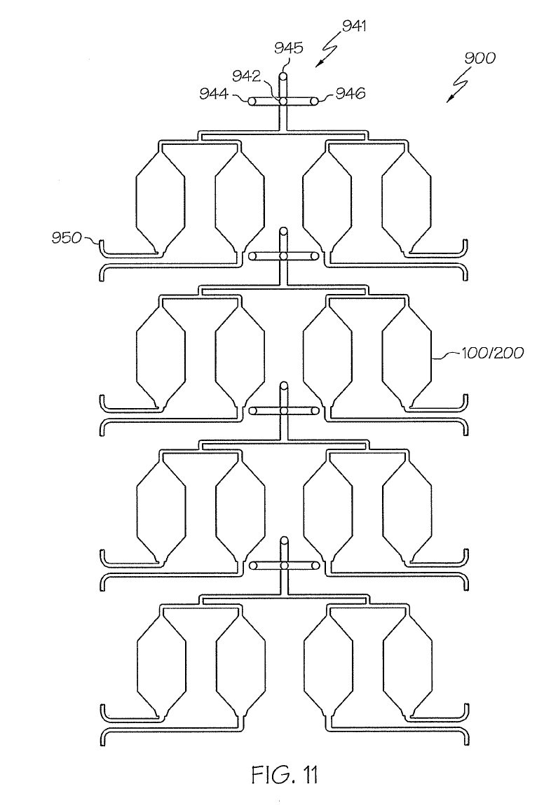

In embodiments, there is provided a system comprising a plurality of the devices 100 and/or 200. The system may be in the fouli of a microtiter plate. It will be appreciated however, that the skilled artisan may arrange the devices 100 and/or 200 to any desired configuration. An exemplary arrangement is shown in FIG. 11. The system 900 comprises a plurality of devices 100 and/or 200. The plurality of devices 100 and/or 200 of system 900 may share inlet 941 where inlet 941 is in fluid communication with a plurality of chambers. The shared inlet 941 may comprise a valve 942 that may be connected with or attached to or built within the inlet 941. The valve 942 may be in fluid communication with a reservoir solution through at least one tube or channel. By way of non-limiting example, FIG. 11 shows valve 942 connected to three tubes or channels 944, 945 and 946. It will be appreciated that any number of tubes may be attached to valve 942 depending on the desired use for the system 900. Valve 942 may be connected between the inlet 941 and at least one tube connecting with a solution reservoir (i.e. 944, 945 and 946). Micropumps may be used to generate through the valve/inlet a fluidic flow within the chamber of the devices. Alternatively, devices 100 and/or 200 of system 900 may each independently have at least two inlets (for example, see FIGS. 1 and 2) and one outlet 950 with each inlet being in fluid communication with a reservoir.

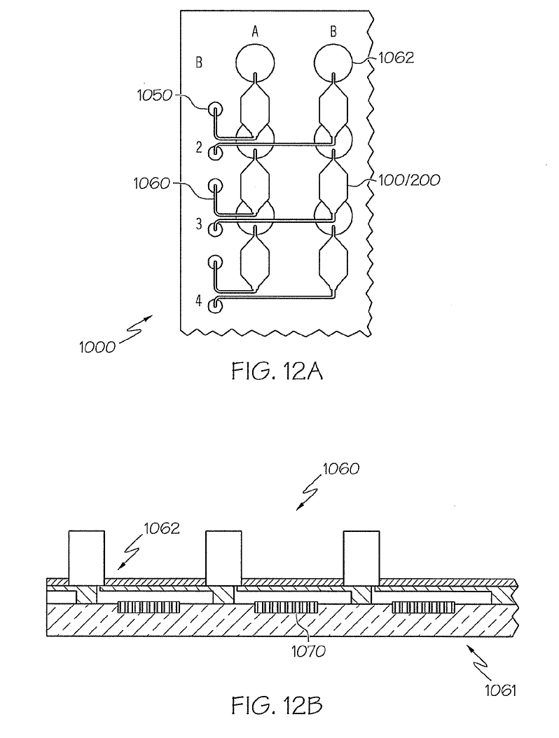

In embodiments, there is provided a multiwell plate system 1000 (FIGS. 12a and 12b) which may comprise the devices 100 and/or 200 integrated with a standard sized microwell plate 1060. As shown in FIG. 12a, the multiwell plate system 1000 may comprise a standard sized multiwell plate 1060 having a planar bottom layer 1061 with the devices 100 and/or 200. As illustrated in FIG. 12b, a biosensor 1070 is integrated within the bottom layer 1061, wherein a chamber covers the biosensor with the biosensor itself formulating the bottom of the chamber. On the top of the chamber, there is a plurality of wells 1062 connecting with the inlet of the biosensor-chamber system 100 or 200. The plurality of wells 1062 of the multiwell plate 1060 may be in fluid communication with the inlet ports of the devices 100 and/or 200. The wells 1062 may act as reservoirs for the devices 100 and/or 200. Additionally, the devices 100 and/or 200 may comprise outlets 1050 which may be positioned at the edge of microplate 1060 within the footprint of the multiwell plate. Although each of the devices 100 and/or 200 is shown in FIG. 12a as independently comprising an outlet 1050, two or more of the devices 100 and/or 200 may share an outlet 1050. It will be appreciated that while only six wells 1062 are depicted, any number of wells 1062 may be present as desired by the skilled artisan. In a non-limiting example, the wells 1062 may be arranged in standard spacing patterns of 96, 384 or 1536 multiwell plates. The use of such standard multiwell plates may allow utilization of conventional robotic systems for automated processing of high throughput cell culture experiments.

In embodiments, the biosensor may be capable of being used in label-free cell based assays. Label-free cell-based assays generally employ a biosensor to monitor ligand-induced responses in living cells. A biosensor typically utilizes a transducer such as an optical, electrical, calorimetric, acoustic, magnetic, or like transducer, to convert a molecular recognition event or a ligand-induced change in cells contacted with the biosensor into a quantifiable signal. These label-free biosensors can be used for molecular interaction analysis, which involves characterizing how molecular complexes form and disassociate over time, or for cellular response, which involves characterizing how cells respond to stimulation. The biosensors of the devices may be, but not limited to, optical biosensor systems such as surface plasmon resonance (SPR) and resonant waveguide grating (RWG) biosensors and optical interferometer-based biosensor, and electric biosensor systems such as bioimpedance systems.

SPR relies on a prism to direct a wedge of polarized light, covering a range of incident angles, into a planar glass substrate bearing an electrically conducting metallic film (e.g., gold) to excite surface plasmons. The resultant evanescent wave interacts with, and is absorbed by, free electron clouds in the gold layer, generating electron charge density waves (i.e., surface plasmons) and causing a reduction in the intensity of the reflected light. The resonance angle at which this intensity minimum occurs is a function of the refractive index of the solution close to the gold layer on the opposing face of the sensor surface.

An RWG biosensor can include, for example, a substrate (e.g., glass) with an embedded grating structure, a waveguide thin film and a cell layer. The RWG biosensor utilizes the resonant coupling of light into a waveguide by means of a diffraction grating, leading to total internal reflection at the solution-surface interface, which in turn creates an electromagnetic field at the interface. This electromagnetic field is evanescent in nature, meaning that it decays exponentially from the sensor surface; the distance at which it decays to 1/e of its initial value is known as the penetration depth and is a function of the design of a particular RWG biosensor, but is typically on the order of about 200 nm. This type of biosensor exploits such evanescent waves to characterize ligand-induced alterations of a cell layer at or near the sensor surface.

RWG instruments can be subdivided into systems based on angle-shift or wavelength-shift measurements. In a wavelength-shift measurement, polarized light covering a range of incident wavelengths with a constant angle is used to illuminate the waveguide; light at specific wavelengths is coupled into and propagates along the waveguide. Alternatively, in angle-shift instruments, the sensor is illuminated with monochromatic light and the angle at which the light is resonantly coupled is measured. The resonance conditions are influenced by the cell layer (e.g., cell confluency, adhesion and status), which is in direct contact with the surface of the biosensor. When a ligand or an analyte interacts with a cellular target (e.g., a GPCR, a kinase) in living cells, any change in local refractive index within the cell layer can be detected as a shift in resonant angle (or wavelength).

The Corning® Epic® system uses RWG biosensors for label-free biochemical or cell-based assays (Corning Inc., Corning, N.Y.). The Epic® System consists of an RWG plate reader and SBS (Society for Biomolecular Screening) standard sized microtiter plates, each well of which contains an RWG biosensor in the bottom. The detector system in the plate reader exploits integrated fiber optics to measure the shift in wavelength of the incident light, as a result of ligand-induced changes in the cells. A series of illumination-detection heads are arranged in a linear fashion, so that reflection spectra are collected simultaneously from each well within a column of a 384-well microplate. The whole plate is scanned so that each sensor can be addressed multiple times, and each column is addressed in sequence. The wavelengths of the incident light are collected and used for analysis. A temperature-controlling unit can be included in the instrument to minimize spurious shifts in the incident wavelength due to the temperature fluctuations. The measured response represents an averaged response of a population of cells.

Electrical biosensors consist of a substrate (e.g., plastic), an electrode, and a cell layer. In this electrical detection method, cells are cultured on small gold electrodes arrayed onto a substrate, and the system's electrical impedance is followed with time. The impedance is a measure of changes in the electrical conductivity of the cell layer. Typically, a small constant voltage at a fixed frequency or varied frequencies is applied to the electrode or electrode array, and the electrical current through the circuit is monitored over time. The ligand-induced change in electrical current provides a measure of cell response. Impedance measurement for whole cell sensing was first realized in 1984. Since then, impedance-based measurements have been applied to study a wide range of cellular events, including cell adhesion and spreading, cell micromotion, cell morphological changes, and cell death. Classical impedance systems suffer from high assay variability due to use of a small detection electrode and a large reference electrode. To overcome this variability, the latest generation of systems, such as the CellKey system (MDS Sciex, South San Francisco, Calif.) and RT-CES (ACEA Biosciences Inc., San Diego, Calif.), utilize an integrated circuit having a microelectrode array.

Optical biosensor imaging systems, including SPR imaging system, ellipsometry imaging, and RWG imaging system, offer high spatial resolution, and are preferably used in the disclosure. For example, SPR Imager®II (GWC Technologies Inc) uses prism-coupled SPR, and takes SPR measurements at a fixed angle of incidence, and collects the reflected light with a CCD camera. Changes on the surface are recorded as reflectivity changes. Thus SPR imaging collects measurements for all elements of an array simultaneously.

Alternatively, a swept wavelength optical interrogation system based on RWG biosensor for imaging-based application may be employed. In this system, a fast tunable laser source is used to illuminate a sensor or an array of RWG biosensors in a microplate format. The sensor spectrum can be constructed by detecting the optical power reflected from the sensor as a function of time as the laser wavelength scans, and analysis of the measured data with computerized resonant wavelength interrogation modeling results in the construction of spatially resolved images of biosensors having immobilized receptors or a cell layer. The use of image sensor naturally leads to an imaging based interrogation scheme. 2 dimensional label-free images can be obtained without moving parts.

Alternatively, an angular interrogation system with transverse magnetic or p-polarized TM₀ mode can also be used. This system consists of a launch system for generating an array of light beams such that each illuminates a RWG sensor with a dimension of approximately 200 μm×3000 μm or 200 μm×2000 μm, and a CCD camera-based receive system for recording changes in the angles of the light beams reflected from these sensors. The arrayed light beams are obtained by means of a beam splitter in combination with diffractive optical lenses. This system allows up to 49 sensors (in a 7×7 well sensor array) to be simultaneously sampled at every 3 seconds.

Alternatively, a scanning wavelength interrogation system can also be used. In this system, a polarized light covering a range of incident wavelengths with a constant angle is used to illuminate and scan across a waveguide grating biosensor, and the reflected light at each location can be recorded simultaneously. Through scanning, a high resolution image across a biosensor can also be achieved.

Cells are dynamic objects with relatively large dimensions, e.g., typically tens of microns. RWG biosensors enable detection of ligand-induced changes within the bottom portion of cells, determined by the penetration depth of the evanescent wave. Furthermore, the spatial resolution of an optical biosensor is determined by the spot size (about 100 microns) of the incident light source. Thus, a highly confluent cell layer is generally used in order to achieve optimal assay results; and the sensor configuration can be viewed as a three-layer waveguide composite, including, for example, a substrate, waveguide thin film, and a cell layer. Following a 3-layer waveguide biosensor theory in combination with cellular biophysics, we found that for whole-cell sensing, a ligand-induced change in effective refractive index, the detected signal ΔN, is governed by equation (1):

$$\begin{matrix}Δ N = S ( N ) Δ {n}_{C} \\ = S ( N ) α d {\sum }_{i} Δ {C}_{i} [ {e}^{\frac{- {z}_{i}}{Δ {Z}_{C}}} - {e}^{\frac{- {z}_{i + 1}}{Δ {Z}_{C}}} ]\end{matrix} \qquad ( 1 )$$

where S(C) is the system sensitivity to the cell layer, and Δn_{c} is the ligand-induced change in local refractive index of the cell layer sensed by the biosensor. ΔZ_{c} is the penetration depth into the cell layer, α is the specific refractive index increment (about 0.18/mL/g for proteins), zᵢ is the distance where the mass redistribution occurs, and d is an imaginary thickness of a slice within the cell layer. Here the cell layer is divided into an equally-spaced slice in the vertical direction. We assumed that the detected signal is, to a first order, directly proportional to the change in refractive index of the bottom portion of cell layer Δn_{c}. The Δn_{c} in turn is directly proportional to changes in local concentration of cellular targets or molecular assemblies within the sensing volume, given the refractive index of a given volume within cells is largely determined by the concentration of biomolecules, mainly proteins. A weighted factor exp(−zᵢ/ΔZ_{c}) is taken into account for a change in local protein concentration that occurs, considering the exponentially decaying nature of the evanescent wave. Thus, the detected signal is the sum of mass redistribution occurring at distinct distances away from the sensor surface, each with unequal contribution to the overall response. Eq. (1) suggests that the detected signal with an RWG biosensor is sensitive primarily to the vertical mass redistribution, as a result of a change in local protein concentration. The detected signal is often referred to as a dynamic mass redistribution (DMR) signal.

In a typical impedance-based cell assay, cells are brought into contact with a gold electrode arrayed on the bottom of culture wells. The total impedance of the sensor system is determined primarily by the ion environment surrounding the biosensor. Under application of an electrical field, the ions undergo field-directed movement and concentration gradient-driven diffusion. For whole cell sensing, the total electrical impedance has four components: the resistance of the electrolyte solution; the impedance of the cell; the impedance at the electrode/solution interface; and the impedance at the electrode/cell interface. In addition, the impedance of a cell comprises two components: the resistance; and the reactance. The conductive characteristics of cellular ionic strength provide the resistive component, whereas the cell membranes, acting as imperfect capacitors, contribute a frequency-dependent reactive component. Thus, the total impedance is a function of many factors, including, for example, cell viability, cell confluency, cell numbers, cell morphology, degree of cell adhesion, ionic environment, the water content within the cells, the detection frequency, and like considerations. Therefore, a bioimpedance signal of living cells upon stimulation is also an integrated cellular response.

Because of its integrated nature, a biosensor output signal such as a DMR signal can be used to study cell signaling under different stimulation conditions.

The disclosure further provides methods for monitoring cell signaling under pulse stimulation. The methods of the disclosure use microfluidics or fluidics to control the environment surrounding cells, or control the duration and times of stimulus exposed to the cells for studying cellular behaviors and functions, such as, but not limited to, cell signaling and ligand pharmacology. In one aspect, the microfluidics or fluidics may be operated at relatively low or non-existent laminar flow, such that the flow itself does not trigger any cellular responses, thus minimizing interference of the flow with the stimulus-induced responses. When employed, one important aspect of laminar flow in microfluidic devices is that it allows fluids to flow side by side for long distances without mixing, which is a common feature in biological systems.

In embodiments, a method is provided to deliver a stimulus to the cells in a controlled manner to provide pulse stimulation. The method may comprise the steps of introducing at least one cell to a chamber of a device wherein the device comprises a biosensor, the biosensor comprising a top surface, the chamber covering the biosensor and the chamber comprising an interior surface including a bottom surface, wherein the bottom surface of the chamber is or is adjacent to the top surface of the biosensor, at least one inlet in fluid communication with the interior surface of the chamber, the inlet comprising a valve wherein the valve is in fluid communication with at least two reservoir solutions, and at least one outlet in fluid communication with the interior surface of the chamber. The method further comprises the steps of allowing at least one cell to attach to the bottom surface of the chamber, contacting the cell with a first solution, wherein the first solution flows through the chamber, contacting the cell with a second solution wherein the second solution flows through the chamber to replace the first solution, and kinetically monitoring the cell response to the first and second solutions. The biosensor, for example, may be a resonant waveguide grating biosensor, a surface plasmon resonance-based biosensor, an optical interferometer-based biosensor, or an electric biosensor.

The first and second solutions may flow through the chamber and contact the at least one cell by being introduced into the chamber through an inlet, flowing through the chamber to an outlet. The chamber may comprise at least two inlets and at least one outlet. Alternatively, the chamber may comprise at least one inlet and outlet, and a valve that may be connected with, attached to or built within the inlet. The valve may be in fluid communication with at least two reservoir solutions through at least two tubes that are attached with respective reservoir of a first solution and a second solution.

The cells may be directly cultured onto the bottom surface of the chamber, or brought to contact with the bottom surface of the chamber. Either way, at least one cell should be attached to the bottom surface of the chamber. After the cells are attached, a first solution is introduced into the chamber, flowing through one of the inlets, contacting the cells and exiting the chamber through the outlet. After a first amount of time, a second solution is introduced into the chamber through a second inlet or through switching to a second reservoir. It may be introduced together with the first solution or the first and second solutions may be introduced sequentially, replacing the first solution in the chamber. The second solution may contact the cells before exiting the chamber through the outlet. After the second amount of time, the first solution may then be reintroduced to the chamber replacing the second solution or the flow of the second solution is stopped. Micropumps may be used to introduce the solutions into the chamber. The flow rate of the solutions may be, by way of non-limiting example, from about 0.5 microliters/min to about 5 microliters/min. It will be appreciated that the first and second solution may be alternated and flowed through the chamber for any number of times. It will be further appreciated that more than two solutions may be introduced into the chamber. By way of non-limiting example, a third solution may be introduced into the chamber replacing the second solution. Alternatively, the first solution may be introduced into the chamber replacing the second solution before introducing the second solution. It will be appreciated that any combination of reservoir solutions may be used by the skilled artisan. In another embodiment, the first and second solutions may be introduced side-by-side through two independent inlets. The two solutions will not mix due to the laminar flow nature within the chamber. After a first amount of time, the flow of the first solution may be stopped with the second solution replacing the first solution over time. The cellular responses, between the regions exposed to the first solution and the second solution may be recorded and compared.

In embodiments, the first solution is a neutral buffer that may have no effect on the cells in the chamber. The second solution may comprise a stimulant, such as a receptor antagonist or agonist, a hormone or a chemical compound, for example a drug candidate. In an alternate exemplary embodiment, the first solution may comprise a stimulant, while the second solution may be a neutral buffer. In yet another exemplary embodiment, the first solution may differ from the second solution by a component such as a chemical or a drug candidate. In yet another exemplary embodiment, more than two solutions can be used in a sequential and multi-step manner. In yet another exemplary embodiment, more than two solutions can be used in a sequential and multi-step manner.

The method of the disclosure further comprises monitoring the cell response to the first and second solutions. As described above, methods for monitoring cell response using biosensors are well known in the art. Examples of monitoring cell responses using RWG are described in PCT/US2006/013539, PCT/US2006/008582 and PCT/US2008/002314, which are hereby incorporated by reference in their entirety. Monitoring the cell response may be done qualitatively or quantitatively. For example, an initial screening of compounds may be done using qualitative monitoring while further studies of compounds of interest from the initial screen may be monitored quantitatively.

The fluidic or microfluidic devices of the disclosure enable the study nonuniformities and complex signaling, such as biological systems in gradients. Nonuniformity is probably the most biologically relevant issue that conventional systems such as microplate-based cell assays can not achieve.

The microfluidics and devices of the disclosure are used to deliver the stimulus to the cells in a controlled manner, such that the cells are only exposed to pulse stimulation. Therefore the cells may be only exposed to a stimulus for a short duration by switching a stimulus solution with a buffered solution. The pulse stimulation can be used to differentiate long-acting ligands from short-acting ligands, to differentiate the short term stimulation-induced cell signaling from the sustained stimulation-induced cellular events. Furthermore, repeated pulse stimulation can also be applied for studying receptor cross-talks, receptor re-sensitization processes, and complexity in pharmacology and functional selectivity of ligands. In addition, the devices may allow constant influx of reagents and removal of by-products. Such ability may be important in studying exocytosis-related cellular events, as well as receptor transactivation. For example, G protein-coupled receptor (GPCR) activation, in many cases, causes transactivation of epidermal growth factor (EGF) receptor through enzymatic releasing of membrane bound EGF ligands. Using constant flow of a solution, the released EGF ligands can be removed, thus the cellular response due to the transactivation can be minimized.

In embodiments, there is provided a method for monitoring mechanical force-induced cell signaling and the subsequent chemical intervention of the mechanical force-induced cell signaling. The method may comprise the steps of introducing at least one cell to a chamber of a device wherein the device comprises a biosensor, the biosensor comprising a top surface, a chamber covering the biosensor, the chamber comprising an interior surface including a bottom surface, wherein the bottom surface of the chamber is or is adjacent to the top surface of the biosensor, at least one inlet in fluid communication with the interior surface of the chamber, the inlet comprising a valve wherein the valve is in fluid communication with at least two reservoir solutions, and at least one outlet in fluid communication with the interior surface of the chamber. The method further comprises the steps of allowing at least one cell to attach to the bottom surface of the chamber, contacting the cell with a first solution, wherein the first solution flows through the chamber at a flow rate wherein the flow rate increases in a step-wise manner, contacting the cell with a second solution wherein the second solution flows through the chamber at a given flow rate, and monitoring the cell response to the first and second solutions. The first solution, flowing at a step-wise increasing flow rate, may result in mechanical force-induced cell signaling. Once the cell signaling is induced, the high flow rate will be maintained, and the second solution, comprising a compound or other moiety, may be introduced into the chamber to contact the cell and the effect of the compound or moiety on the mechanical force-induced cell signal may be determined. Alternatively, a solution containing a chemical or drug candidate molecule may be flowed through the chamber to contact the cell with a flow rate increasing in a step-wise manner, such that the impact of the chemical or drug candidate molecule on the mechanical force induced signaling on the cell can be directly measured. The flow-rate threshold for the mechanical force-induced signaling occurring in the absence and presence of the molecule or the drug candidate molecule may be used as an indicator of the ability of the molecule to intervene in the mechanical sensing of the cells.

### EXAMPLES

The disclosure will be further clarified by the following examples.

### Experimental Procedures

Materials: Epinephrine, norepinephrine, salmeterol, cycloheximide, formeterol, salbutamol, thrombin, and glycerol were obtained from Sigma Chemical Co. (St. Louis, Mo.). SFLLR-amide was obtained from Bachem (King of Prussia, Pa.). Corning® Epic® 384 and 96-well biosensor inserts were obtained from Corning Inc. (Corning, N.Y.) and cleaned by exposure to high-intensity UV light (UVO cleaner, Jelight Co. Inc., Laguna Hills, Calif.) for 6 minutes before use. Corning Epic® 384 well biosensor cell culture compatible microplates were obtained from Corning Inc, and used without any further treatment.

Fabrication of PDMS chambers and assembly of biosensor device: The first step of fabrication consisted in generating a silicon master. This was achieved by U.V. photolithography on a Si [100] wafer. Briefly, 4″ silicone wafers were primed with P-20 immediately before the resist was applied. 1 μm thick Shipley 1813 photoresist was spun on the wafer at 3000 rμm for 30 sec (acceleration 1000 rpm/s) and soft baked on a hot plate for 1 Min at 110° C. The wafers were exposed to UV-light through a chromium mask with the desired structures designed as CAD-drawing using MA6 (Karl Zeiss) mask aligner. After post bake of 2 min at 80° C. the wafers were finally developed (60-100 s, MF-319, Shipley), thoroughly rinsed with water and dried. Molds for 200 μm deep fluidic channels and cell culture chambers were etched into the silicone using Plasma Therm 72 fluorine based reactive ion etcher. After photoresist stripping and cleaning silicone masters were exposed to trichloro(1H, 1H, 2H, 2H)-perfluorooctyl vapor for 2 h for pasivation.

PDMS replicas of structures were produced by pouring a PDMS precursor mixture over the whole 4″ silicon master (1:10 curing agent to prepolymer ratio, Sylgard 184, Dow Corning, US). It was then cured at 70° C. for 80 min. The cured PMDS was peeled of from the silicone mold to complete the fabrication. As a result, PDMS devices having a array of 4×6 chambers, each chamber having one inlet and one outlet, were made.

Once the PDMS chambers were made, these substrates were subject to surface oxidation using O₂ plasma cleaning for 30 sec at pressure of 500 mTorr, and attached onto the biosensor insert such that within a chamber there is a single biosensor locating within the center of the chamber. Afterwards, each chamber was filled with 75% ethanol twice, each for 30 sec, followed by washing with PBS buffer and drying. The array of 4×6 PDMS chamber device was attached onto the 96 well biosensor insert, such that within each chamber there is a RWG biosensor located at the center of the chamber. The 96 well biosensor insert consists of an array of 8×12 biosensors in a footprint that is SBS-compatible 96 well microplate. Each chamber has an inlet, and an outlet. A valve is connected to the inlet, and also with a pump. The biosensor dimension is about 3 mm×3 mm. Each chamber has a dimension of'-9 mm long from the inlet to the outlet, of ±5 mm in width, and ±200 microns in height. Similarly a 384 well biosensor insert can also be used.

Cell culture: All cell lines were obtained from American Type Cell Culture (Manassas, Va.). The cell culture medium used was Dulbecco's modified Eagle's medium (DMEM) supplemented with 10% fetal bovine serum (FBS), 4.5 g/liter glucose, 2 mM glutamine, and antibiotics for both human epidermoid carcinoma A431 and human lung carcinoma A549.

Cells were typically grown using ˜1 to 2×10⁴ cells per well at passage 3 to 15 suspended in 50 μl of the corresponding culture medium in the biosensor microplate, and were cultured at 37° C. under air/5% CO₂ for ˜1 day. Except for A431 which was subject to ˜20 hr starvation through continuously culture in the serum-free DMEM, the A459 cells were directly assayed without starvation. The confluency for all cells at the time of assays was ˜95% to 100%. For cell culture in the microfluidics-biosensor chamber systems, ˜4×10⁴ cells suspended in 10 μl the medium containing 10% FBS were injected into the chamber, and were cultured at 37° C. under air/5% CO₂ until ˜95% confluency was reached (˜1 day). To prevent drying during the culture, the biosensor device was maintained within a petri dish with a cover, and extra cell medium within the dish.

Because the biological status of cells (e.g., cell viability, confluency and degree of adhension) could significantly impact the measurement of mass redistribution, the quality of cells was inspected using both light microscopy and the optical system. The optical system allows one to collect the resonant image of whole sensor at the single cell resolution, such as is described, for example, in co-owned U.S. patent application Ser. No. 12/128,267 to Fang, Y., et al, the disclosure of which is incorporated herein in its entirety.

Characterization of a chemical gradient within the biosensor device: To characterize the biosensor device, the biosensor device was subject to a pulse flow of glycerol and water. The biosensor outputs due to the bulk index change were recorded and analyzed. Alternatively, mathematical modeling was also applied to characterize the flow behavior within the biosensor system.

Label-free biosensor cell assays: Corning RWG (resonant waveguide grating) imaging system was used for label-free biosensor cell assays. The imaging system, based on swept wavelength interrogation, is capable of high resolution imaging, with a spatial resolution of 6 micron in the direction perpendicular to the propagation path of the coupled light, a resolution of about 150 or about 6 microns in the direction parallel to the propagation path of the coupled light. The cellular responses across the whole sensor were recorded in real time at each pixel (6 microns×6 microns). The cellular responses at defined locations or areas of the biosensor surface were averaged to generate responses of a population of cells within the detection area(s).

Alternatively, Corning® Epic® scanning wavelength interrogation system was also used, in conjunction with “buffer swapping” approach to differentiate the long-acting agonists from the short-acting agonists.

The A431 cells reached high confluency before assays, and were subject to starvation for overnight with serum free medium. For A549 cells, cells were grown to high confluency and used for assays without starvation. Before assays, the cell medium was replaced with 1× Hanks' balanced salt solution (HBSS) having 20 mM Hepes buffer, and incubated within the imaging system for about 1 hour. The chamber was filled with 6.6 micro-liters buffered solution. The HBSS buffer solution was initially introduced into the chamber from the inlet and flowed through the biosensor with a low flow rate (typically 1 μl/min or 1 microliter/min) using a controlled pump until a steady baseline was reached. Then, a first solution, typically a solution containing a receptor agonist or a compound dissolved in the HBSS buffer, was introduced by switching the valve, and flowed through the biosensor for a period of time (e.g., 1 min, or 5 min, or 30 min). Subsequently, the HBSS buffer solution was reintroduced and flowed through the biosensor to replace the first solution. After certain time period, a second solution, typically a solution containing a receptor agonist or a compound, either different or the same as the first solution, was introduced and flowed through the biosensor continuously to trigger a cellular response. The flow rate was typically relatively low (˜0.5 μ/min to 5 μl/min), such that no mechanical sensitive cellular responses were triggered, and the cellular responses observed were largely due to the chemical(s) in the solution. Different combinations of these steps can be applied, depending on the purpose of the assays. The cellular responses throughout the assays were recorded using the high resolution imaging system. Depending on the biosensor insert used, the biosensor has a dimension of 3 mm×3 mm or 2 mm×2 mm. The chamber dimension is a length of about 9 mm from the inlet to the outlet, a width of 5 mm, and a height of 100 microns from the sensor surface to the bottom surface of the chamber. The total volume of solution required to fill the chamber is about 6 micro-liters.

The RWG biosensor exploits its evanescent wave to measure ligand-induced dynamic mass redistribution (DMR) signals in cells. The evanescent wave extends into the cells and exponentially decays over distance, leading to a characteristic sensing volume of about 150 nm, implying that any optical response mediated through the receptor activation only represents an average over the portion of the cell that the evanescent wave is sampling. Such sampling with the biosensor is sufficient to differentiate the signaling of distinct classes of receptors in living cells under different stimulation conditions.

Like SPR, the RWG biosensor is sensitive to refractive index—an intrinsic property of biomolecules. Since the refractive index of a given volume within a cell is largely determined by the concentrations of bio-molecules such as proteins, we found, based on a three-layer waveguide grating theory, that a ligand-induced optical response is largely associated with dynamic mass redistribution. The relocation of cellular targets towards the sensor surface (e.g., relocation of intracellular targets to the activated receptors at the basal membrane surface) makes a positive contribution to the DMR (P-DMR); conversely, the movement of cellular targets away from the sensor surface (e.g., receptor internalization) is a negative contributor to the DMR (N-DMR). The aggregation of these events determines the kinetics and amplitudes of a ligand-induced DMR. However, recent studies, using PWR technology and in vitro reconstituted G protein-coupled receptors immobilized onto the sensor surface, showed that a ligand-induced optical response of the receptor-lipid membrane system consists of two components—changes in mass density and changes in structure. Since the RWG biosensor used here is unable to differentiate the contributions of these components, ligand-induced changes in organization of biomolecules in living cells may also contribute to the overall response measured.

For biosensor cellular assays, a baseline was established first. All studies were carried out at controlled temperature (22° C.) and with three replicates for each measurement, unless specifically mentioned.

### Example 1

### The Characterization of a (Micro)Fluidics-RWG Biosensor System

To characterize the flow behavior of the (micro)fluidics-RWG biosensor system, the alternative flow of glycerol solution and water was used and their optical responses were recorded. The biosensor system had the following dimensions: Chamber—two inlets and one outlets; the distance between the inlet and outlet was about 9 millimeters, the width was about 4 millimeters, the height was about 200 microns, and the total volume that filled the chamber was about 6 micro-liters; and the biosensor of 2 millimeters×2 millimeters located within the center of the chamber. Alternatively, an alternative flow of fluorescence dye solution and water was generated and examined using fluorescence microscopy.

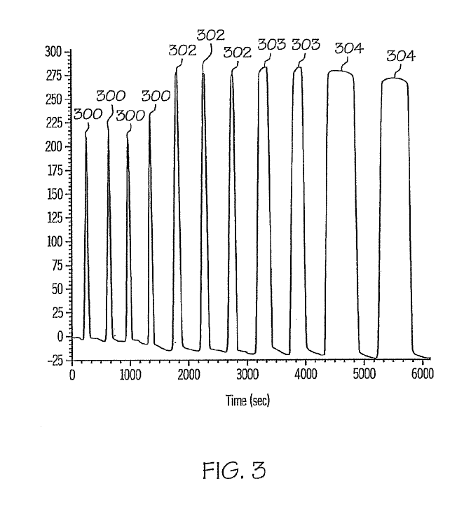

First, the residence time of the injection plugs of different volumes at 1 microliter/min flow rate was monitored. Here a series of 2% glycerol solutions of a given volume were flowed into the chamber between a constant flow of the HBSS buffer. The change in bulk refractive index led to an increase in resonant wavelength, when the glycerol solution was flowed through the chamber. The averaged response across the whole sensor was recorded and plotted as a function of time (FIG. 3). The total volume of glycerol solution injected between the buffer flows were indicated in FIG. 3: 1 microliter (300), 2 microliter (302), 4 microliter (303) and 9 microliter (304).

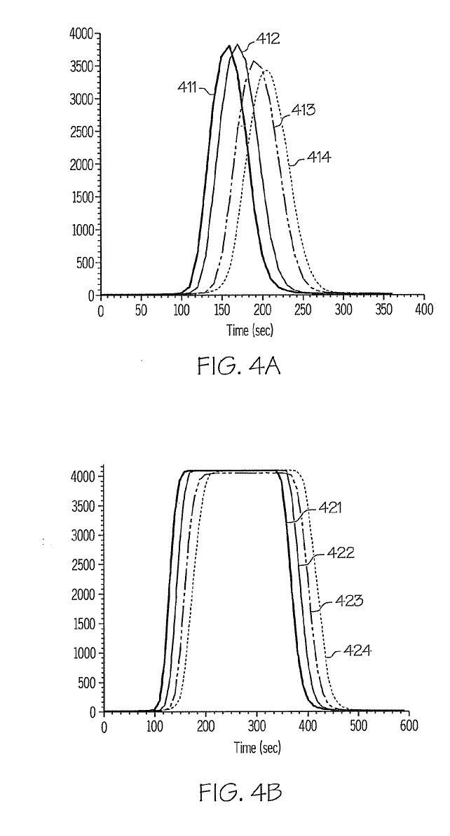

For the fluorescence microscopy observations, sulforhodamine B solution (0.3 mM in HBSS buffer) was injected into microfluidic chamber prefilled with buffer. Since the main interest of concentration profiles relates to the RWG biosensor area, the measurements were done at four different lines spaced 400 μm apart on the biosensor. Sulforhodamine B injection was followed by buffer wash at 1 microliter/min. Typical results of fluorescent intensity above the biosensor are shown in FIG. 4. FIG. 4a shows the fluorescence intensity profiles of 4 different zones across the biosensor: 411, 412, 413, and 414 (from the area closest to the inlet to the area closest to the outlet), upon alternative flows of buffer, dye solution and buffer, with a flow rate of 1 microliter/min, and the volume of dye solution of 1 microliter. FIG. 4b shows the fluorescence intensity profiles of 4 different zones across the biosensor: 421, 422, 423, and 424 (from the area closest to the inlet to the area closest to the outlet), upon alternative flows of buffer, dye solution and buffer, with a flow rate of 1 microliter/min, and the volume of dye solution of 4 microliter.

The comparison of peak shapes, residence time and time delay between different zones was made for fluorescence measurements (Fluorescence) and biosensor imaging system (Optical). The data are summarized in Table 1. Results showed that both methods led to comparable fluidic characteristics within the fluidics-biosensor system.

TABLE 1
Characteristics of flows within the microfluidics-biosensor system.

| | 1 microliter | | 2 microliter | | 4 microliter | | 9 microliter | |
|---|---|---|---|---|---|---|---|---|
| | injection | | injection | | injection | | injection | |
| | Fluorescence | Optical | Fluorescence | Optical | Fluorescence | Optical | Fluorescence | Optical |
| Delay | 16 | 20 | 18 | 20 | 18 | 20 | 17 | 19 |
| between zones | | | | | | | | |
| (sec) | | | | | | | | |
| Peak width at | 55 | 50 | 118 | 100 | 240 | 220 | 540 | 518 |
| ½ height (sec) | | | | | | | | |
| Total peak | 155 | 138 | 235 | 215 | 357 | 330 | 668 | 650 |
| width (sec) | | | | | | | | |
| Peak height | 89 | 79 | 100 | 100 | 100 | 100 | 100 | 100 |
| (% of maximum) | | | | | | | | |

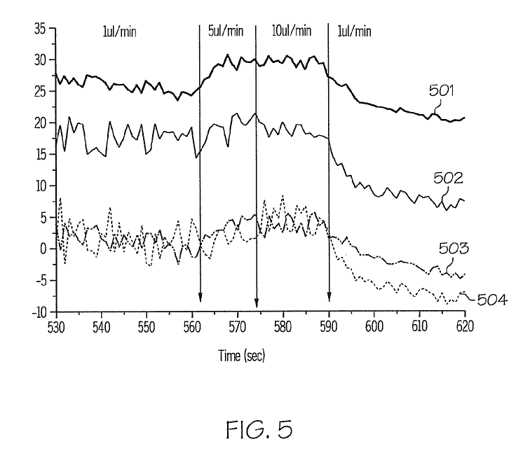

Second, the influence of solution flow on the biosensor measurements, particularly the system background noise as well as temperature fluctuation and/or cellular response to the mechanical force generated by the flow, was also examined. The RWG imaging system was used to real time monitor the biosensor responses upon different combinations of fluidic movement. The main results are shown in FIG. 5. FIG. 5 shows the signal fluctuation of the biosensor system having a layer of quiescent A431 cells upon the fluidic movement of buffer at different flow rate. The different scans, 501, 502, 503, and 504, represent four replicate experiments. For visualization purpose, the signal was not normalized to zero at the staring point. These results indicate that background signals were within the range of variations that are typical for RWG biosensor cell-based assays and the flow rate in the range examined had little impact on the cellular baseline signal. However, at the highest flow rate examined, the cells do give rise to a detectable response, possibly associated with the mechanical force-induced signaling of cells. Therefore, for the following studies, low flow rates were typically used for studying ligand pharmacology and receptor biology.

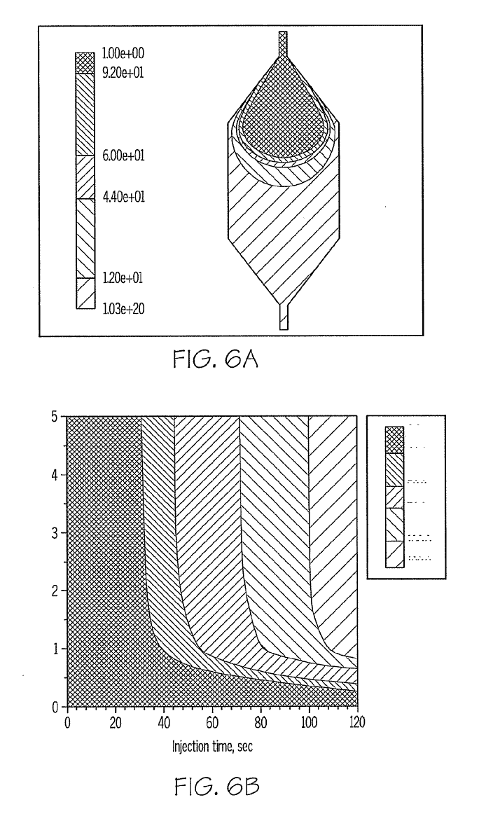

Since the duration of cells upon stimulation with a compound is important in studying receptor transduction and understanding drug pharmacology, a fluid dynamics simulation was run on a fluidics-biosensor-cell system. Such modeling allows the prediction of fluid flows, residence time and shear stresses inside the chamber upon injection of different volumes of compounds at different flow rates. To simulate fluidic flow, CFD package Fluent 6.3 was used to numerically solve the steady state Navier-Stokes equations. Standard pressure-velocity coupling scheme was SIMPLE. Fluid properties were set to those of HBSS buffer. HBSS buffer was modeled as an incompressible, isothermal, Newtonian fluid. Domain was defined as a two-dimensional plane. Velocity inlet boundary conditions were used; outlet boundary condition was a zero pressure outflow; the walls were “no-slip” walls. Boundary layer was meshed starting at 1 micron with 1.2 ratio and 43 microns deep. It was found that simulation results are highly dependent on the refinement of model mesh. Final model mesh contained 167600 finite volume mesh elements. Further increase in the number of cells rendered identical simulation results. Typical simulation results were shown in FIG. 6. FIG. 6a shows 30 sec tracer injection at 2 microliter/min flow rate. Results indicated that the edge of fluid movement is mostly not straight, and the center area of chamber from the inlet to the outlet is more appropriate for data acquisition and analysis. Simulation results obtained were compared to the one obtained with fluorescence dye injection experiment. Nonetheless, since the biosensor dimension is much smaller than that of each chamber, and each biosensor is located within the center of each chamber, the fluidic dynamics can be considered as constant and the fluidic flow is uniform on the biosensor area, given that the flow rate is within the range examined.

Next, theoretical time exposure of the cells that are located on the biosensor was calculated at different flow rates and different injection volumes. FIG. 6b shows the map of cell treatment time as a function of injection volume and flow rate. Exposure time can be effectively controlled either by volume of the injected compound or by flow rate if different shear stress conditions are needed.

### Example 2

### Buffer Swapping Approach for Studying Long-Acting Agonists for Beta2-Adrenergic Receptors

Current biosensor assays generally measure cellular responses under sustained stimulation with compounds—a compound is added into a well having cells contacted with the sensor surface by pipetting or using an onboard or offline liquid handling device, and it remains in the solution throughout the assay. To study the drug pharmacology, particularly the ability of ligands (agonists or antagonists) for long-acting through a receptor, a buffer swapping approach was developed. Here, shortly after a ligand solution was introduced to the well having cells cultured on the sensor surface, the ligand solution was quickly removed through either an automated pipettor or simply flicking. Afterwards, a buffer solution was added back to cover the cells. Due to the cell breathing and/or cellular responses to such buffer swapping, the cells were maintained within the buffer for a period of 30 min to 60 min. Finally, a second agonist solution was introduced. The impact of the initial pulse stimulation with the first ligand on the second agonist-induced biosensor output signal can be used as an indicator for the ability of the first ligand for long-acting through the receptor both ligands targeted.

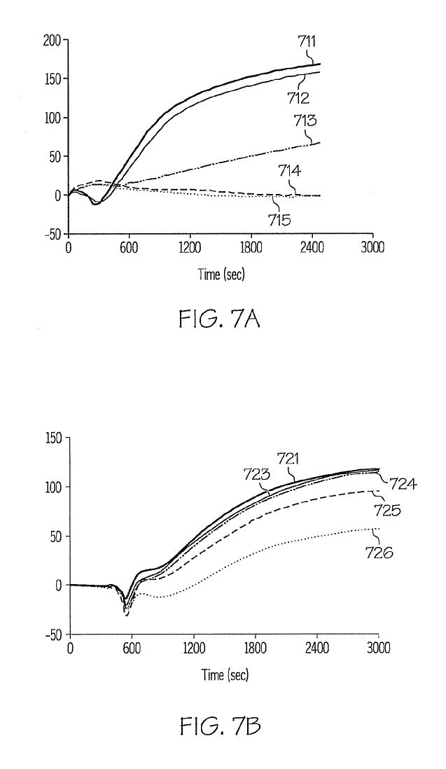

Using such approach, panels of β2-adrenergic receptor (β2AR) agonists were surveyed. Results were summarized in FIG. 7. FIG. 7a showed the 2 nM epinephrine-induced DMR signal of quiescent A431 cells, in which the cells were pre-treated with a pulse stimulation with different agonists for about 150 sec: buffer (711), 2 nM epinephrine (712), 0.5 nM salmeterol (713), 1 nM CGP12177 (714), and 0.1 nM formeterol (715). Here, epinephrine and formeterol are full agonists for the β2AR, whereas salmeterol of 0.5 nM and CGP 12177 are partial agonists to the β2AR. Except for epinephrine, all other three agonists are long-acting agonists for the PAR. Results showed that the 150 sec pulse stimulation of cells with both CGP12177 and formeterol inhibited the epinephrine response, whereas salmeterol of 0.5 nM partially attenuated the epinephrine response. Conversely, the short-acting agonist epinephrine did not cause the de-sensitization of the cells to the second stimulation with epinephrine. This is contradictory to that obtained under sustained stimulation condition—A431 cells pre-treated with 2 nM epinephrine become completely desensitized to the second stimulation with 2 nM epinephrine (data not shown). These results suggest that CGP 12177, formeterol or salmeterol, but not epinephrine, act as long-acting agonists, and the pulse stimulation, through the buffer swapping approach, can be used to determine whether a ligand is long-acting or not.

FIG. 7b shows the 2 nM epinephrine-induced DMR signal of quiescent A431 cells, in which the cells were pre-treated with a pulse stimulation with different agonists for about 60 sec: buffer (721), 2 nM epinephrine (723), 100 nM norepinephinre (724), 100 nM salbutamol (725), and 100 nM salmeterol (726). Here, norepinephrine is a strong partial but short-acting agonists for the β2AR, whereas salmeterol of 100 nM are a full agonist but long-acting agonist to the β2AR. Results showed that such short-term pulse stimulation of cells with salmeterol of 100 nM partially inhibited the epinephrine response, whereas others had little impact on the epinephrine response. These results indicate that salmeterol indeed is a long-acting agonist, and the duration of initial pulse stimulation is important for the sensitivity of such assay for determining long-acting agonism of a ligand for the β2AR. Nonetheless, these results suggest that such buffer swapping approach allows the differentiation of long-acting agonism, at least for β2AR.

### Example 3

### Pulse Stimulation-Induced DMR Signals of Quiescent A431 Cells

The abovementioned buffer swapping approach disallows the direct measures of cellular responses under pulse stimulation. This is partly because of buffer swapping approach triggered unwanted cellular responses, beside the pulse stimulation. Such unwanted response, typically lasting for about 30 min, interferes with the label-free biosensor measures of the pulse-stimulation-induced response.

Here, the microfluidics-biosensor system was used to directly measure the pulse stimulation-induced cell signaling. To do so, the microfluidics-biosensor system having a confluent layer of quiescent A431 cells was pre-filled with 1×HBSS buffer. A constant flow of HBSS buffer was replaced with an agonist solution for 2 min, and followed by switching back with the buffer solution. All solutions were at a flow rate of 1 microliter/min.

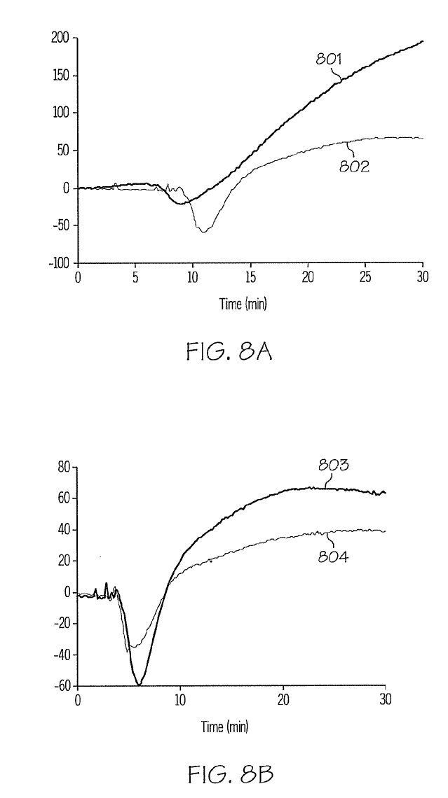

FIG. 8 shows the DMR signals of quiescent A431 cells under pulse stimulation with different agonists: 2 nM epinephrine (802, and 803) and 100 nM salbutamol (804), in comparison with the epinephrine response under sustained stimulation condition (801) in which a 2 nM epinephrine solution was flowed across the bio sensor throughout the assay. For pulse stimulation, a duplicate of epinephrine responses were obtained and found to be reproducible. Results showed that the sustained epinephrine response under the slow flow rate is comparable to that obtained using microplate-based sustained response (711 in FIG. 7). In addition, under pulse stimulation, both epinephrine and salbutamol led to a much smaller response. This is probably due to that under such short duration both short-acting agonists are unable to trigger sustained cellular response, particularly for G protein-independent signaling which typically takes place much slower after stimulation, compared to the G protein-dependent signaling.

### Example 4

### Pulse Stimulation-Induced Label-Free Optical Signals of A549 Cells

Protease activated receptors (PARs) consist of a family of four G protein-coupled receptors (GPCRs) which to date include PAR₁, PAR₂, PAR₃ and PAR₄. PARs are found in a large variety of normal and malignant tissues and cells including skin, platelets, endothelial cells, gastrointestinal tract, brain and lungs. Instead of being activated through reversible ligand binding, PARs utilize a unique proteolytic mechanism for activation. Serine proteases such as thrombin and trypsin site-specifically cleave the receptor within the extracellular N-terminal exodomain. The activating cleavage site is the residue 41-42 (R↓SFLLRN), 36-37 (R↓SLIGKV), 38-39 (K↓TFRGAP) and 47-48 (R↓GYPGQV) for human PAR₁, PAR₂, PAR₃ and PAR₄, respectively. The cleavage unmasks a new N-terminus, which, in turn, acts as a tethered ligand sequence. The tethered ligand domain binds intramolecularly to and activates the receptor, thus initiating signaling. The proteases that activate PARs include coagulation factors (e.g. thrombin, coagulation factors VIIa and Xa), proteases from inflammatory cells (e.g., mast cell tryptase, neutrophil cathepsin G) and enzymes from epithelial tissues (e.g., trypsins). PAR₁, PAR₃ and PAR₄ are activated principally by thrombin, while PAR₂ is activated by trypsin-like proteases such as mast cell tryptase and coagulation Factor Xa. Synthetic PAR-activating peptides (PAR-APs), corresponding to the first five or six amino acids of the tethered ligand sequences, for example SFLLR-amide, can directly activate PARs, except for PAR₃. Since these synthetic peptides function as receptor agonists independent of proteolysis, PAR-APs are useful for studying the physiological and pathophysiological functions of PARs. Although these agonist peptides appear to elicit full responses, there is an emerging body of evidence which suggests that agonist peptides do not activate PAR₁ in the same manner as does thrombin.

Classical receptor theory presents a model in which a single functional response is intrinsically associated with a given GPCR. However, there is growing evidence that diverse signaling responses can be invoked by single GPCRs in response to interaction with divergent ligands. This phenomenon of “agonist trafficking” or “ligand-induced functional selectivity” or “functional selectivity” stands in stark contrast to conventional receptor pharmacology.

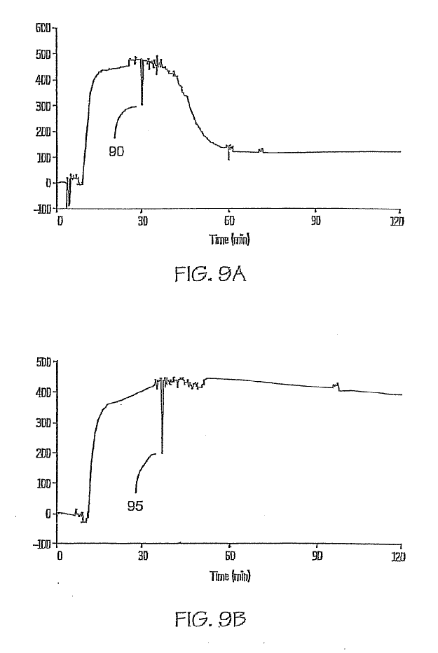

Receptors such as the serotonin, β2-adrenergic, dopamine, octopamine, and others continue to expand the diversity of receptor families which demonstrate functional selectivity. However, conventional cell assays are typically carried out under “sustained” simulation conditions. Thus, these cellular assays are difficult to differentiate functional selectivity of various ligands. As shown in FIGS. 9a and 9b, two PAR₁ receptor agonists, thrombin at 5 unit/ml (FIG. 9b) and SFLLR-amide at 10 μM (FIG. 9a), both at a concentration of EC₁₀₀, triggered an almost indistinguishable DMR signal in A549 cells—both agonists led to a P-DMR signal with a maximal of amplitude of ˜400 picometers in terms of shift in resonant wavelength of the biosensor, as measured using the RWG biosensor under a constant flow of 1 microliter per min.

By replacing the agonist solution 30 min after stimulation with the HBSS buffer, the present biosensor cellular assays directly enable the differentiation of cell signaling mediated by thrombin or SFLLR-amide. After switching back to HBSS, the SFLLR-amide treated cells responded to a slowly decaying signal down to a plateau level that is about 120 picometers above the baseline, suggesting that a large amount of increased mass within the detection zone of the biosensor are moved away from the biosensor surface (FIG. 9a). In contrast, after switching back to the HBSS, the thrombin treated cells still maintained elevated signal (FIG. 9b). This result suggests that the thrombin triggered cellular response is largely irreversible, consistent with the fact that thrombin cleaves the N-terminal of the PAR1 receptor, leading to receptor degradation. Conversely, the SFLLR-amide triggered cellular response is largely reversible, consistent with the fact that SFLLR-amide binds to and activates the PAR1, leading to the receptor internalization and recycling. After removal of SFLLR-amide, the cells are able to return their inactivated state.

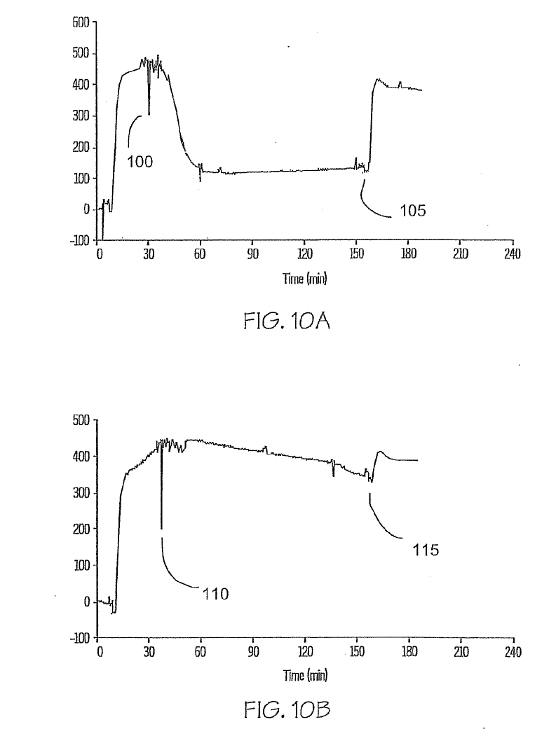

To further confirm these findings, a second repeated stimulation with thrombin at 10 unit/ml was examined after the initial pulse stimulation. Results are shown in FIGS. 10a and 10b. Here the A549 cells were first exposed to either SFLLR-amide (10 μM) (FIG. 10a) or thrombin (5 unit/ml) (FIG. 10b) for 30 min. HBSS buffer was then introduced into the chamber to replace the agonist solution and continuously flowed across the biosensor for ˜2 hours. Afterwards, a second stimulation with 10 unit/ml thrombin was carried out by continuously flowing the thrombin solution across the biosensor. Throughout the assays, all solution flow rates were about 1 microliter per min. Results showed that the SFLLR-amide treated cells were still able to respond to the second stimulation with thrombin with slightly smaller amplitude (FIG. 10a), while the thrombin treated cells only gave rise to a little response upon repeated stimulation with the second thrombin exposure (FIG. 10b). These results suggest that the SFLLR-amide treated cells can become resensitize, probably due to the recycling of internalized receptors back to the cell surface. On the other hand, the thrombin treated cells largely lost the ability to become resensitize, due to the degradation of the internalized receptors induced by thrombin.

To further elucidate the cellular mechanism accounting for the little response of A459 cells upon repeated stimulation with the second thrombin stimulation, 10 μM of cycloheximide was included in each solution, the HBSS buffer solution, the first agonist solution (SFLLR-amide or thrombin), and the second agonist thrombin solution, to ensure that there are equal amount of cycloheximide throughout the assay. Cycloheximide is an inhibitor of protein biosynthesis in eukaryotic organisms. Cycloheximide exerts its effect by interfering with the translocation step in protein synthesis (movement of two tRNA molecules and mRNA in relation to the ribosome) thus blocking translational elongation. Cycloheximide is widely used in biomedical research to inhibit protein synthesis in eukaryotic cells studied in vitro. It is inexpensive and works rapidly. Its effects are rapidly reversed by simply removing it from the culture medium. Results showed that the presence of cycloheximide largely eliminated the second response of the thrombin-treated A549 cells, but not the SFLLR-amide treated cells, upon repeated stimulation with the second thrombin solution (data not shown). These results indicate that after thrombin treatment, A549 cells lost almost all of their cell surface PAR₁ receptors, and the second response (i.e., FIG. 10b) of the thrombin pretreated cells is largely due to the de novo synthesis of PAR₁ receptors.

Taken together, these results indicate that the present pulse stimulation biosensor cellular assays using the (micro)fluidics-biosensor systems of the disclosure enable the study of receptor resensitization and desensitization process, and also the differentiation of ligand directed functional selectivity, particularly for these that are difficult to be manifested using conventional cellular assays.

It will be apparent to those skilled in the art that various modifications and variations can be made to the disclosure without departing from the spirit and scope of the invention. Thus it is intended that the disclosure cover the modifications and variations provided they come within the scope of the appended claims and their equivalents.

What is claimed is:

1. A device for monitoring the response of at least one cell to pulse-stimulation, the device comprising:
a biosensor, the biosensor comprising a top surface;
a chamber covering the biosensor, the chamber comprising an interior surface including a bottom surface, wherein the bottom surface of the chamber is, or is adjacent to, the top surface of the biosensor, at least one inlet in fluid communication with the interior surface of the chamber, the inlet comprising a valve, wherein the valve is in fluid communication with at least two reservoir solutions, and at least one outlet in fluid communication with the interior surface of the chamber.

2. The device of claim 1 wherein the biosensor is a resonant waveguide grating biosensor, a surface plasmon resonance-based biosensor, an optical interferometer-based biosensor, or an electric biosensor.

3. The device of claim 1 further comprising a micropump, wherein the micropump is in fluid communication with the inlet.

4. The device of claim 1 wherein the chamber has a width of about 500 microns to about 10 millimeters.

5. The device of claim 1 wherein the chamber has a width of from about 1 millimeter to about 5 millimeters.

6. The device of claim 1 wherein the chamber has a height of from about 200 microns to about 2 millimeters.

7. The device of claim 1 wherein the valve is in fluid communication with at least three solution reservoirs.

8. The device of claim 1 wherein the system comprise at least two inlets.

9. A multiwell plate system comprising a plurality of the devices of claim 1 and a multiwell plate.

10. A method to provide pulse stimulation to cells comprising the steps of:
introducing at least one cell to a chamber of a device, wherein the device comprises a biosensor, the biosensor comprising a top surface, a chamber covering the biosensor, the chamber comprising an interior surface including a bottom surface, wherein the bottom surface of the chamber is, or is adjacent to, the top surface of the biosensor, at least one inlet in fluid communication with the interior surface of the chamber, the inlet comprising a valve wherein the valve is in fluid communication with at least two reservoir solutions, and at least one outlet in fluid communication with the interior surface of the chamber; allowing at least one cell to attach to bottom surface of the chamber;
contacting the cell with a first solution for a first amount of time, wherein the first solution flows through the chamber;
contacting the cell with a second solution for a second amount of time, wherein the second solution flows through the chamber replacing the first solution; and
monitoring the cell response to the first and second solutions.

11. The method of claim 10 further comprising the step of contacting the cell with a third solution for a third amount of time after the second solution, wherein the third solution flows through the chamber replacing the second solution.

12. The method of claim 11 further comprising the step of contacting the cell with the first solution for a period of time wherein the first solution flows through the chamber replacing the second solution, before contacting the cell with the third solution.

13. The method of claim 10 wherein a common molecule is included in all the solutions.

14. The method of claim 10 wherein the second solution comprises a cell stimulating factor.

15. The method of claim 10 further comprising the step of contacting the cell with the first solution again after the second solution, wherein the first solution flows through the chamber replacing the second solution.

16. The method of claim 10 further comprising repeating the steps of contacting the cell with the first and second solutions, wherein the steps are repeated at least one time.

17. The method of claim 10 wherein the first and second solutions flow through the chamber at a flow rate of from about 0.5 microliters/min to about 5 microliters/min.

18. The method of claim 10 wherein the biosensor is a resonant waveguide grating biosensor, a surface plasmon resonance-based biosensor, an optical interferometer-based biosensor, or an electric biosensor.

19. A method for monitoring mechanical force-induced cell signaling comprising the steps of:
introducing at least one cell to a chamber of a device wherein the device comprises a biosensor, the biosensor comprising a top surface, a chamber covering the biosensor, the chamber comprising an interior surface including a bottom surface, wherein the bottom surface of the chamber is, or is adjacent to, the top surface of the biosensor, at least one inlet in fluid communication with the interior surface of the chamber, the inlet comprising a valve wherein the valve is in fluid communication with at least two reservoir solutions, and at least one outlet in fluid communication with the interior surface of the chamber; allowing at least one cell to attach to bottom surface of the chamber;
contacting the cell with a first solution, wherein the first solution flows through the chamber at a stepwise increasing flow rate; and
monitoring the cell response to the first solution for mechanical force-induced cell signaling.

20. The method of claim 19 further comprising the steps of:
contacting the cell with a second solution comprising a compound, peptide, protein, hormone or other moiety after mechanical force-induced cell signaling is detected, wherein the second solution flows through the chamber; and
monitoring the cell response to the second solution, the flow rate of the first solution is below the threshold flow rate that causes the cell detachment, and the biosensor is a resonant waveguide grating biosensor, a surface plasmon resonance based biosensor, an optical interferometer based biosensor, or an electric biosensor.
